(19) United States
(12) Patent Application Publication
(10) Pub. No.: US 2006/0064174 A1
(43) Pub. Date: Mar. 23, 2006

(54) Implantable valves and methods of making the same

(76) Inventor: Reza Zadno, Fremont, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 10/948,731

(22) Filed: Sep. 22, 2004

(51) Int. Cl. A61F 2/04; A61F 2/06; A61F 2/24

(52) U.S. Cl. 623/23.68; 623/1.24; 623/2.18

(57) ABSTRACT

An implantable valve composed of a frame structure monolithically formed and covered with a biocompatible coating or soft structure. The implantable valve can have various shapes, openings, anchoring sites, attachment sites and is flexible, expendable, and easy to attach to body tissue. In some embodiments, the implantable valve is made by first determining a two or three dimensional configuration of the frame structure, which has an open end and a tapered end. The configuration may be scaled to obtain a desired size, e.g., length and diameter. One or more frame structure may be cut, stamped, etched, or machined from a single material, which could be metal or plastic with various thickness profiles and may have superelasticity and/or shape memory. The biocompatible coating selectively seals the valve to control/prevent fluid passage. To enhance bonding, the frame structure surface is treated with etching, polishing, sand blasting, plating, nanotechnology surface modification, etc.

### BACKGROUND OF THE INVENTION

1. Field of the Invention

This invention relates generally to valves implantable in a hollow organ or vessel such as the heart or a vein or other body cavities. More particularly, it relates to new and improved implantable valves and methods of making the same, each embodiment thereof is composed of a frame structure formed from a single piece of material and a biocompatible protective coating or soft structure.

2. Description of the Related Art

Natural valves in the human body as well as other animals have important functions such as controlling blood flow in the venous system, preventing back flow, controlling blood flow from the atrium to the ventricle and into the arterial system, preventing uncontrolled flow from the bladder, and air flow through the pulmonary system or in the gastro-intestinal system.

Intricately situated, these natural valves are supposed to respond to pressure, or the lack thereof, and control/prevent the flow of fluid passing through it accordingly by folding or closing. However, for various reasons, they often fail to function properly or stop working altogether. As one skilled in the art knows, abnormal, diseased, non-functioning natural valves can lead to many serious complications, ranging from urinary incontinence to blood pumping insufficiency.

Various therapeutic techniques and medical devices, such as open surgery and implantation of artificial valves, are currently used to treat and/or replace failed natural valves. Unfortunately, these prior artificial valves themselves may fail or malfunction for various reasons. For example, artificial valves and valve structures often integrate or join rigid and soft segments, sections, or parts. As such, they are quite susceptible to improper integration, which may result in poor support of the valve opening, shorter fatigue life, and other drawbacks known in the art. In addition, they are typically manufactured from plastics or from a metallic frame that encloses/encapsulates a plastic inner member. Plastics tend to lose integrity, particularly mechanical integrity over time, after many cycles at body temperature, and therefore are not very desirable especially in fatigue or high stress applications.

As one skilled in the art knows, most prior artificial valves are neither suitable for nor can be retrofitted with desirable advanced technologies such as dipping, insert molding, nanotechnology surface modifications. Most prior artificial valves also lack adequate metallic areas and/or anchoring means for proper attachment. As a result, they require undesirable complex suturing and relying heavily on the suturing techniques or careful placement of individual surgeons.

What is more, compared to natural valves, prior artificial valves are quite bulky, thus preventing them from being introduced easily in a percutaneous fashion. Clearly, there is a continuing need in the art for new and improved implantable valves that overcome the aforementioned drawbacks of prior artificial valves and valve structures, that utilize advanced nanotechnology, and that can be introduced with minimal invasiveness. The present invention addresses this need.

### BRIEF SUMMARY OF THE INVENTION

An important goal of the present invention is to provide a viable alternative/replacement to prior artificial valves and valve structures that suffer from various drawbacks as discussed above. This goal is achieved in an implantable valve that is composed of a frame structure monolithically formed from a single piece of material and covered with a biocompatible coating or soft structure.

The frame structure has a customizable open end and a tapered end. The customizable open end can have various shapes, anchoring sites, attachment sites, and so on. The customizable open end can be patterned or otherwise configured such that it is flexible and/or expendable. The tapered end has a plurality of tapered members, panels, or elements that respectively gradually narrow to a common point and that are selectively sealed by the biocompatible coating to control (two-way valve) or prevent (one-way valve) fluid passage.

The frame structure is made from a single piece of material, such as metal or synthetic material made from the polymerization of organic compounds. The frame structure material preferably has memory, for instance, elastic or heat-recoverable shape memory. Suitable materials include stainless steel, Nitinol, Nitinol alloys, nickel-based alloys, cobalt-chromium-nickel alloys, Ni—Ti, Ni—Ti—Nb, Ni—Ti—Mo, Ni—Ti—V, Ni—Ti—Fe, Ni—Ti—Cu, Ni—Ti—Cr, shape memory alloys, copper-based shape memory alloys, polycarbonate, polypropylene, and shape memory plastics. Various manufacturing processes as well as surface treatment/modifications and other techniques may be utilized to form and finalize the frame structure.

A biocompatible coating, covering, or a soft structure is then coated, laminated, bonded, or otherwise applied to the final frame structure. Suitable materials include silicones, polyvinyl, polyether-based polyamides, thermoplastic elastomers, polyurethane, polyethylene, anti-blood clotting coatings, anti-thrombogenic coatings, bioactive coatings, and heparin coatings. To enhance and/or strengthen the bonding, surface treatment and/or modification such as etching, polishing, sand blasting, plating, nanotechnology smart molecule bonding, and other techniques, could also be applied.

In some embodiments, the implantable valve according to the present invention is produced by the following steps:
a) determining a two or three dimensional configuration of a three dimensional frame structure, the configuration includes an open end of the frame structure and a tapered end of the frame structure;
b) scaling the configuration to a desired size;
c) forming the frame structure according to the configuration in step a) or b); and
d) applying a biocompatible coating or soft structure to the frame structure.

In some embodiments, the frame structure is cut, stamped, etched, machined, or otherwise created from a substantially flat (i.e., two-dimensional, e.g., a sheet) or a tubular (i.e., three-dimensional, e.g., a hollow cylinder) material. Alternatively, the frame structure is monolithically formed utilizing injection molding, insert molding, or other precision molding processes. One skilled in the art will appreciate that, compared to the manufacturing processes common in fabricating prior artificial valves, the manufacturing processes necessary to produce the implantable vales according to the present invention are easier, more efficient, and very cost effective.

It is important to note that implantable valves according to the present invention can be readily scaled and are not limited by design. The embodiments described herein may vary in size and configuration according to needs and applications and are limited only by the underlying manufacturing processes employed.

Moreover, in some embodiments, the implantable valve can be rolled, folded, or otherwise reduced to an even more compact size. This advantageously enables the implantable valve to be introduced/delivered percutaneously with minimal invasiveness, for instance, via a catheter, which is highly desirable in the field.

The customizable open end of the implantable valve can also be tailored or otherwise configured in various ways to suit or adapt to different needs and applications. For example, it may have built-in anchoring and/or attachment sites, advantageously eliminating or significantly reducing the need for complex suturing.

Other advantages of the present invention will become apparent to one skilled in the art upon reading and understanding the preferred embodiments described below with reference to the following drawings.

### BRIEF DESCRIPTION OF THE DRAWINGS

FIG. 1 shows a first embodiment of an implantable valve according to the present invention, the implantable valve having a frame structure with an open end and a tapered end.

FIG. 2 shows the frame structure of FIG. 1 covered with a biocompatible coating.

FIG. 3 shows a second embodiment of the implantable valve according to the present invention.

FIG. 4 shows a first substantially flat frame structure material having different patterns and various depth profiles thereof according to the present invention.

FIG. 5 shows a third embodiment of the implantable valve and steps of forming the same according to the present invention.

FIG. 6 shows a fourth embodiment of the implantable valve and steps of forming the same according to the present invention.

FIG. 7 shows a fifth embodiment of the implantable valve and steps of forming and delivering the same according to the present invention.

FIG. 8 shows a second substantially flat frame structure material and steps of forming the same according to the present invention.

FIG. 9 shows a sixth embodiment of the implantable valve and steps of forming the same according to the present invention.

FIG. 10 shows a seventh embodiment of the implantable valve and steps of forming the same according to the present invention.

FIG. 11 shows an eighth embodiment of the implantable valve and steps of forming the same according to the present invention.

FIG. 12 shows a ninth embodiment of the implantable valve and steps of forming the same according to the present invention.

### DETAILED DESCRIPTION OF THE PREFERRED EMBODIMENTS

In the following detailed description, like reference numbers are used to refer to identical, corresponding or similar features and elements in various exemplary embodiments shown in the drawings.

As one skilled in the art knows, natural valves vary in sizes and applications. Similarly, embodiments of the implantable valve according to the present invention vary in sizes and applications. For example, for most arterial applications, the diameter of the open end of the frame structure might vary from about 4 mm to 25.5 mm. Because the implantable valve according to the present invention is significantly more efficient and compact than most prior artificial valves, embodiments implementing the present invention can be readily scaled to match the size of natural valves in various applications.

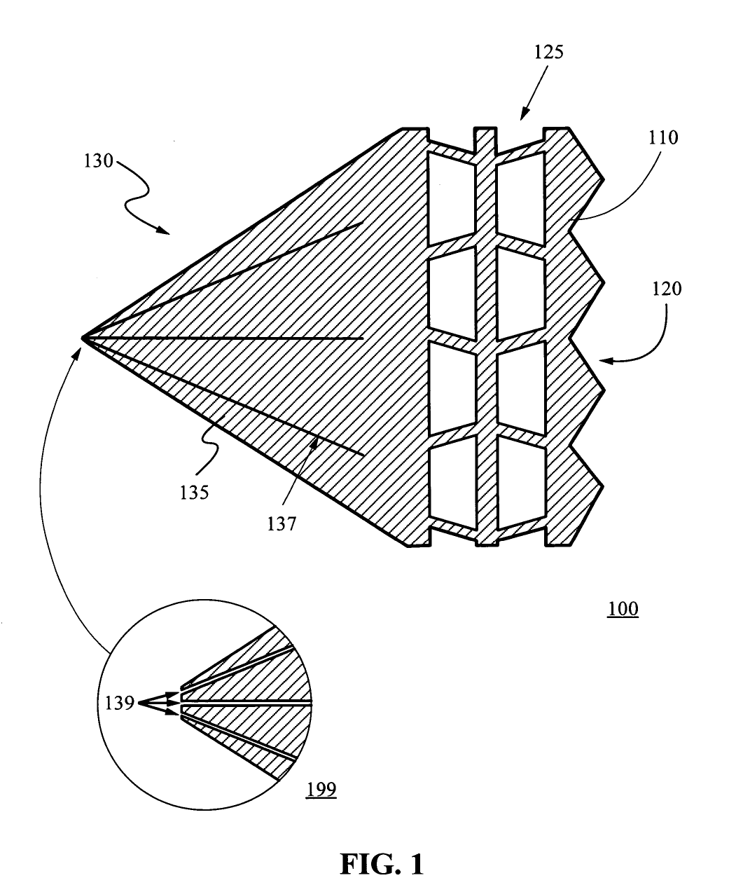

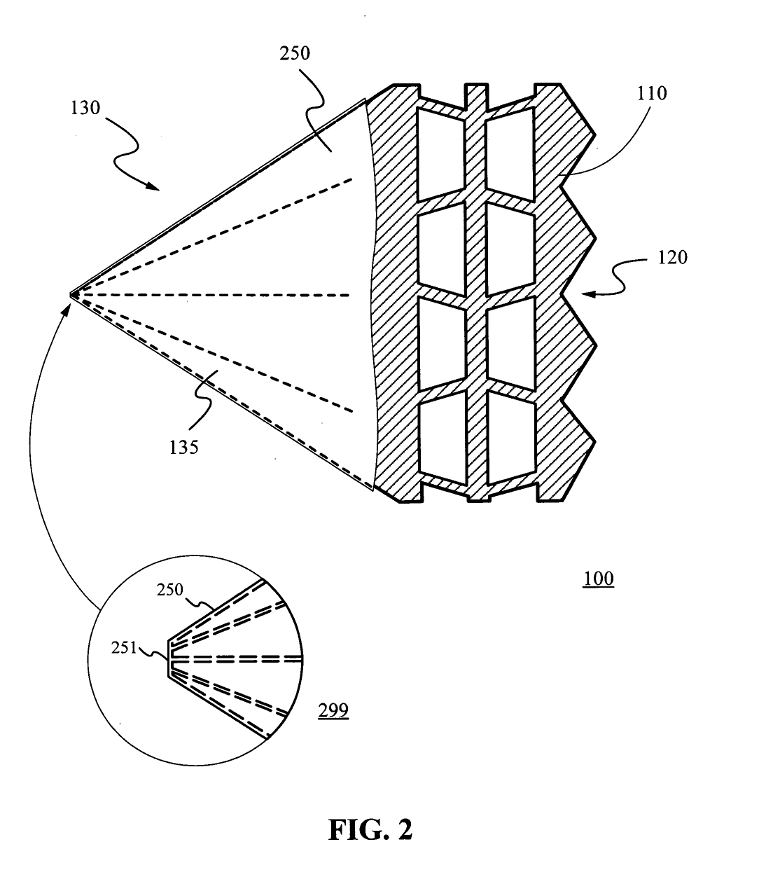

FIGS. 1-2 show a first embodiment of an implantable valve 100 composed of a frame structure 110 monolithically formed from a single piece of material and a biocompatible coating 250. According to the present invention, the single piece of material could be metal or a synthetic material made from the polymerization of organic compounds. The frame structure material preferably has memory, for instance, elastic or heat-recoverable shape memory. Shape memory effect describes the process of restoring the original shape of a plastically deformed material by heating it. This is a result of a crystalline phase change known as “thermoelastic martensitic transformation”.

Materials suitable for implementing the frame structure of the present invention include, but not limited to, nickel-based alloys such as NITINOL (an acronym for Nickel Titanium Naval Ordnance Laboratory), cobalt-chromium-nickel alloys such as Elgiloy®, metallic and plastic shape memory materials, stainless steel, polyether-block co-polyamide polymers such as Pebax®, polycarbonate, polypropylene, and the likes. Elgiloy® is a registered trademark of Elgiloy Limited Partnership for a proprietary cobalt-chromium-nickel alloy often used in highly corrosive environments and with high temperatures. Pebax® resins are available from Atofina Chemicals, Inc. of Philadelphia, Pa. Also known as polyether block amides, polyether-block co-polyamide polymers refer to a family of thermoplastic, melt-processible, polyether-based polyamide that have good hydrolytic stability and are available in a broad range of durometers (stiffnesses) and compositions. This broad range allows applications incorporating radius, pierce/notch, taper, sealing, shaping, and joining modifications in various geometries and material systems for device components.

One skilled in the art will appreciate that the present invention is not limited to the frame structure materials listed herein. With advances in material science continue to be made, other suitable frame structure materials might become available to implement the present invention.

Various manufacturing processes as well as surface treatments/modifications and other techniques may be utilized to form and finalize the frame structure. These enabling processes include, but not limited to, for instance, molding, insert molding, stamping, etching, plasma etching, laser machining, coining, rolling, swaging, deep drawing, adhesive bonding, dipping, coating, laminating, nanotechnology surface modification and molecular bonding, and the likes. One skilled in the art will appreciate that other enabling technologies and processes are possible to manufacture embodiments of the implantable valve according to the present invention.

Preferably, surface treatment and/or modification such as etching, polishing, sand blasting, plating, and other techniques, are applied prior to the dipping, coating, laminating, bonding, or nanotechnology molecule bonding process to enhance/strengthen the bonding between the frame structure and the biocompatible coating.

Materials suitable for implementing the biocompatible coating of the present invention include, but not limited to, anti-thrombogenic coatings, active coatings such as P15, heparin coatings, silicone, thermoplastic elastomers such as C-Flex®, polyurethane, polyethylene, nylon, and the likes. One skilled in the art will recognize that other suitable biocompatible coating materials could also be used to implement the present invention. C-Flex® thermoplastic elastomers are available from Consolidated Polymer Technologies (CPT), Inc. of Clearwater, Fla. A thermoplastic elastomer is defined as a tough, electrically insulating elastomer, with many of the physical properties of vulcanized rubbers but which can be processed as a thermoplastic material. Most thermoplastic elastomers are two-phase systems that have hard and soft phases as known in the art.

Returning now to FIGS. 1-2, the frame structure 110 has a first portion characterized by a customizable open end 120 and a second portion characterized by a tapered end 130. The customizable open end 120 can have various shapes, anchoring sites, attachment sites, and so on. In this exemplary embodiment, the customizable open end 120 has a circular configuration and is designed with a pattern 125 to allow flexibility. As will be further described below, the customizable open end 120 can be patterned or otherwise configured in all conceivable ways to allow flexibility and/or expendability. In some embodiments, the first portion has a flexibility that is different from that of the second portion.

The tapered end 130 has a plurality of tapered members, panels, or elements 135 that respectively gradually narrow to a common point 251. In this embodiment, these tapered members 135 are defined by a plurality of slits or cuts 137 that produce a plurality of substantially small gaps 139 between the tapered members 135, as shown in the exploded view 199.

In FIG. 2, the frame structure 110 is, entirely or a portion thereof, coated, bonded, or otherwise covered with the biocompatible coating 250 to provide both better biocompatibility and sealing for fluid passage. Other suitable bonding processes include, but not limited to, dipping, shrinking, adhesive bonding, laminating, etc. To enhance the bonding, surface treatment/modifications such as etching, polishing, sand blasting, plating, nanotechnology smart molecule bonding, and so on, could also be applied.

As shown in the exploded view 299, the small gaps 139 are selectively sealed by the biocompatible coating 250, enabling the valve, once it is implanted in a hollow organ or vessel such as the heart or a vein, to control (two-way) or prevent (one-way) the flow of blood or fluid passing through it.

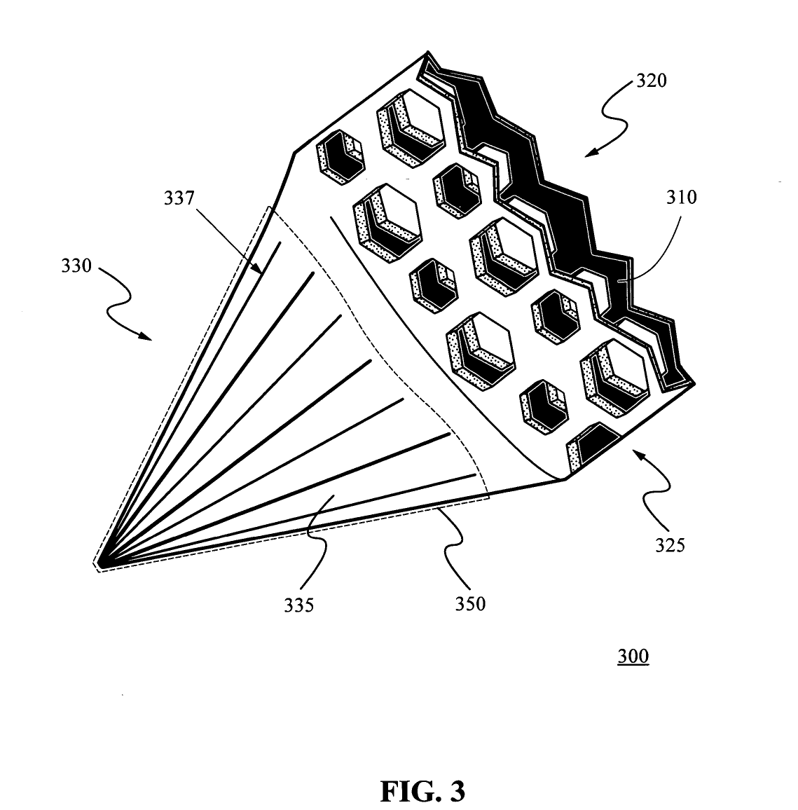

FIG. 3 illustrates a second embodiment 300 according to the present invention. Similar to the first embodiment, the implantable valve 300 comprises a frame structure 310 coated with a biocompatible coating 350, the cover area of which is indicated by dashes. The frame structure 110 has a customizable open end 320 and a tapered end 330. The customizable open end 320 is configured with a pattern 325 to allow more flexibility. The tapered end 330 has a plurality of tapered members 335 defined by a plurality of cuts 337 that are sealed by the biocompatible coating 350.

In some embodiments, the implantable valve according to the present invention is produced by the following steps:
a) determining a two or three dimensional configuration of a three dimensional frame structure, the configuration includes an open end of the frame structure and a tapered end;
b) scaling the configuration to a size suitable for a particular implantation application;
c) forming the frame structure according to the configuration in step a) or, when size adjustment is applicable, according to the scaled configuration in step b); and
d) applying a biocompatible coating or soft structure to the frame structure.

In some embodiments, step d) could be performed before step c). In embodiments with stainless steel frames, for example, a flat sheet of stainless steel could be coated prior to forming it into a cylindrical shape.

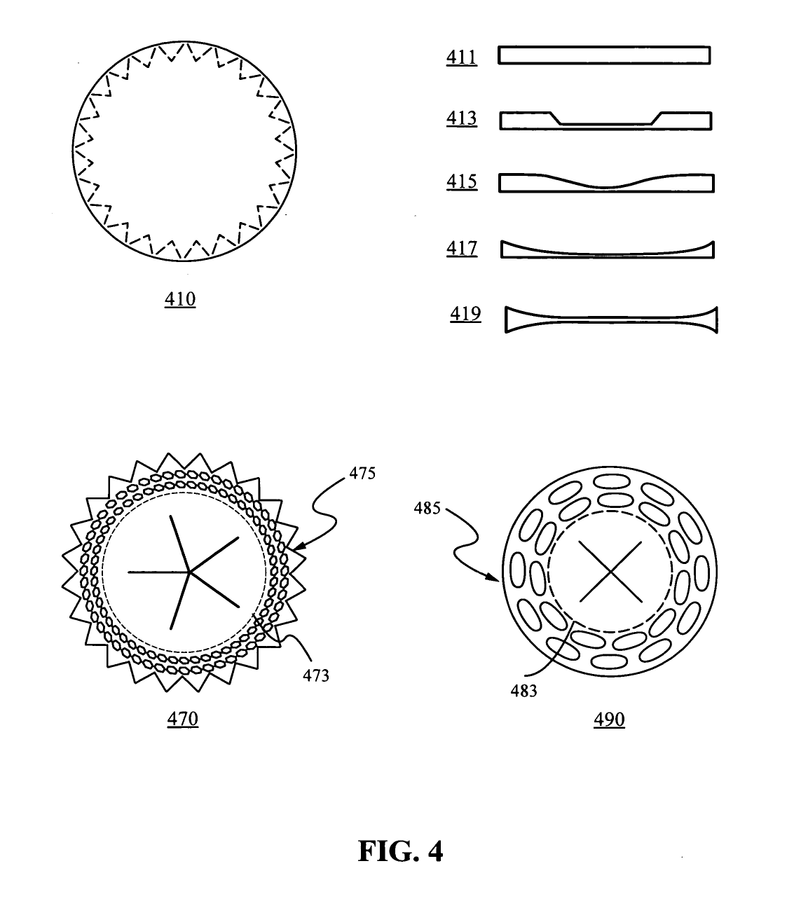

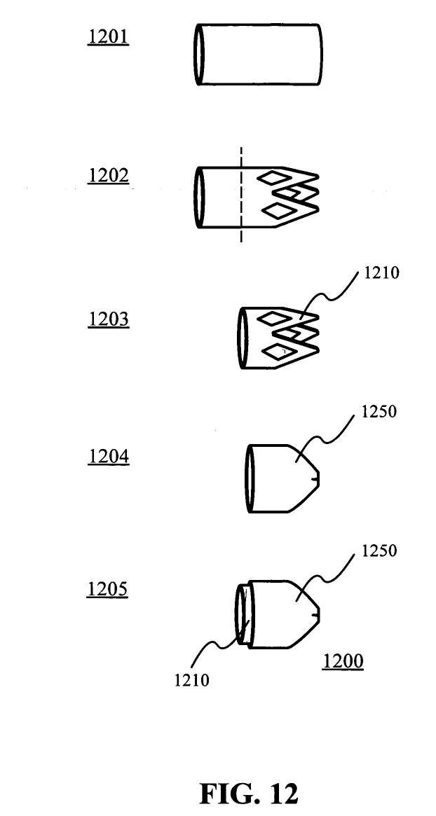

In some embodiments, the frame structure is cut, stamped, etched, machined, or otherwise created from a substantially flat (i.e., two-dimensional, e.g., a sheet, see, FIGS. 4-11) or a tubular (i.e., three-dimensional, e.g., a hollow cylinder, see, FIG. 12) material. As one skilled in the art will appreciate, more than one frame structures having the same or different configurations could be formed at substantially the same time from a single piece of material in substantially one step. For example, a plurality of frame structures could be laser machined from a sheet or tube of a shape memory alloy.

The frame structure could also be monolithically formed utilizing injection molding, insert molding, or other precision molding processes. One skilled in the art will appreciate that, compared to the manufacturing processes common in fabricating prior artificial valves, the manufacturing processes necessary to produce the implantable vales according to the present invention are much easier, more efficient, and very cost effective.

FIG. 4 shows a substantially flat frame structure material 410 that can be stamped with a pattern 475 and several slits joining at the center thereof to form a flat frame structure 470. The dash line 473 indicates approximately where the open end of the frame structure 470 is to end and where the tapered end thereof is to begin. Similarly, the frame structure material 410 can be etched with a pattern 485 and central opening cuts to form a flat frame structure 490. The dash line 483 indicates approximately where the open end of the frame structure 470 is to end and where the tapered end thereof is to begin. In some embodiments, coining is applied to a flat frame structure between the dash line and edges thereof to reduce thickness and to facilitate reducing strain, which is helpful for fatigue testing.

The frame structure material 410 may have depth or thickness profiles 411, 413, 415, 417, and 419 suitable for different applications. These profiles may be achieved via various techniques and processes such as etching and laser machining. As one skilled in the art will appreciate, appropriate thickness may differ from material to material and from application to application. In some embodiments, the preferred range is from about 0.003″ to about 0.010″ for stainless steel, from about 0.005″ to about 0.020″ for Ni-based alloys, and from about 0.010″ to about 0.020″ for non-metallic materials such as polycarbonate.

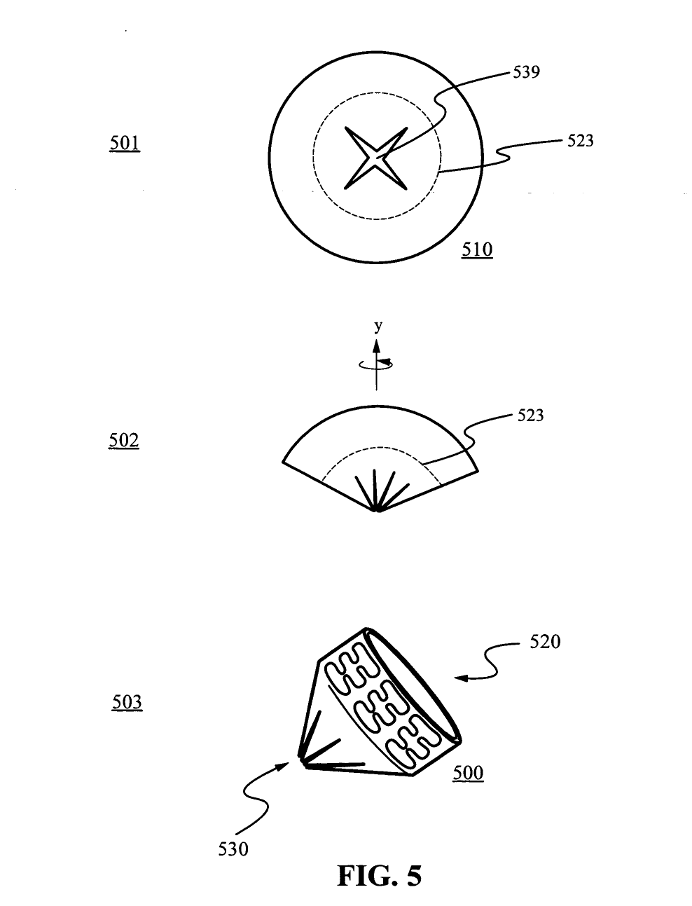

FIG. 5 shows a third embodiment of the implantable valve and steps of forming the same according to the present invention. As shown in step 501, the implantable valve 500 is formed from a flat frame structure 510, which is stamped with a pattern (omitted here for clarity) and slits forming an opening 539. The dash line 523 illustratively separates the edges, which forms the open end 520 in step 503, from the opening 539, which forms the tapered end.

The flat frame structure 510 is rolled up or otherwise turned into a conical shape in step 502 by, for example, sliding it over a mandrel (not shown) and heat set in step 503 at a temperature above 300° C. and mostly at 500° C. for a period of one minute to 30 minutes for shape memory alloys.

One skilled in the art will readily appreciate that different material requires different temperature and time to set and/or cure. For example, Nitinol is a family of inter-metallic materials that contain a nearly equal mixture of nickel (55 wt. % Ni) and titanium (Ti). Other elements can be added to adjust or “tune” the material properties.

In some embodiments, binary high nickel (50.8% at weight of Ni)_Ti is cold worked 20 to 40% and heat-treated about 1-2 minutes at 500° C., about 20-30 minutes at about 350° C., or another suitable combination to achieve superelasticity. Heat treatments for other shape memory materials such as plastics, nickel-based shape memory alloys (e.g., Ni—Ti, Ni—Ti—V, Ni—Ti—W, Ni—Ti—Fe, Ni—Ti—Cr, Ni—Ti—Mo, and Ni—Ti—Cu), and iron-based shape memory alloys are known in the art and thus are not further described herein for the sake of brevity. Alloys with Co could enhance physical properties and could be implemented in this application.

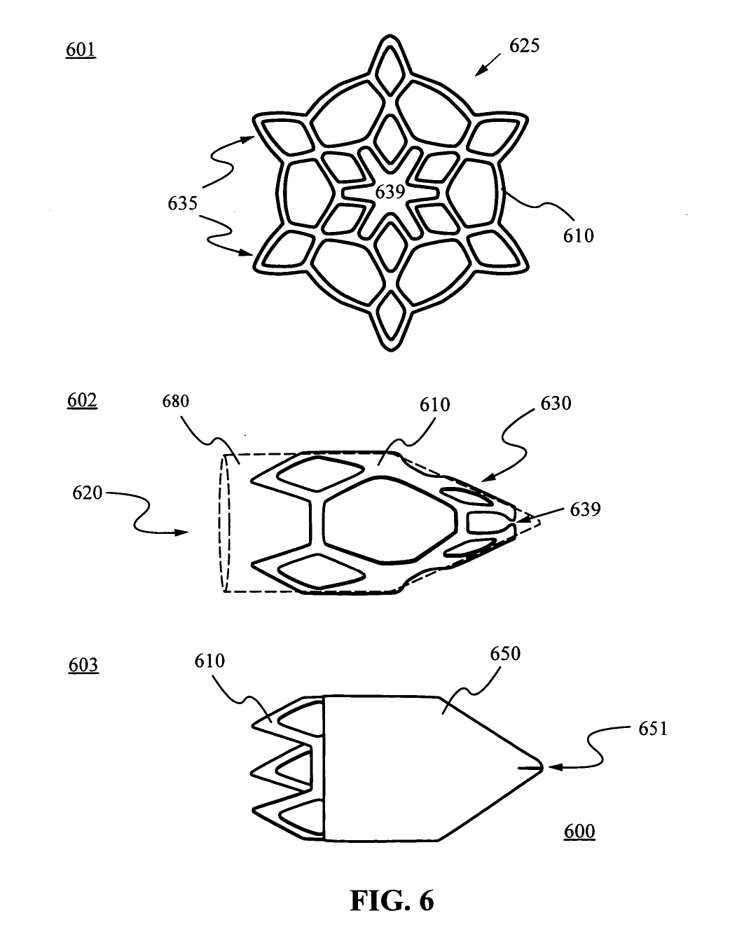

FIG. 6 shows a fourth embodiment of the implantable valve and steps of forming the same according to the present invention. In step 601, the implantable valve 600 is formed from a substantially flat frame structure 610, which is cut, stamped, laser cut, etched, or molded from a variety of materials, some of which have shape memory and some do not. The frame structure 610 has an intricate pattern 625 with multiple openings including a central opening 639 and a plurality of attachment sites 635.

As described above, the frame structure material itself could be substantially flat or tubular. In the latter case, it is possible to carve, etch through, or create cavities inside and around the tubular material and then slice the frame structure 610 alone with the pattern 625. Alternatively, it is possible to slice pieces from the tubular material and then stamp, cut, etch, or laser machine the pattern 625 thereof respectively.

In step 602, the flat frame structure 610 is rolled up or otherwise shaped by, for example, sliding over a mandrel 680 to form an open end 620 and an tapered end 630. The mandrel is preferably a heat-resistant metallic mandrel that does not react to the frame structure material of the frame structure 610. In the case of Nitinol, the sheet metal or tube is heated from about 350° C. to about 600° C. between one and 30 minutes, depending on its starting state. The surface of the frame structure 610 is preferably treated and/or modified prior to step 603.

In step 603, a biocompatible coating 650 is applied to the frame structure 610 by, for example, dipping or shrinking. Suitable dipping materials include silicone, polyurethane, and the likes.

Suitable shrinking materials include PE, PU, C-Flex®, and the likes. As discussed above, other biocompatible coating materials are possible. The biocompatible coating 650 selectively seals the multiple openings of the frame structure 610. A threshold (tip) 651 controls (two-way valve) or prevent (one-way valve) fluid passage. The configuration of the threshold 651 may vary depending on the coating material used and the particulars of a certain application.

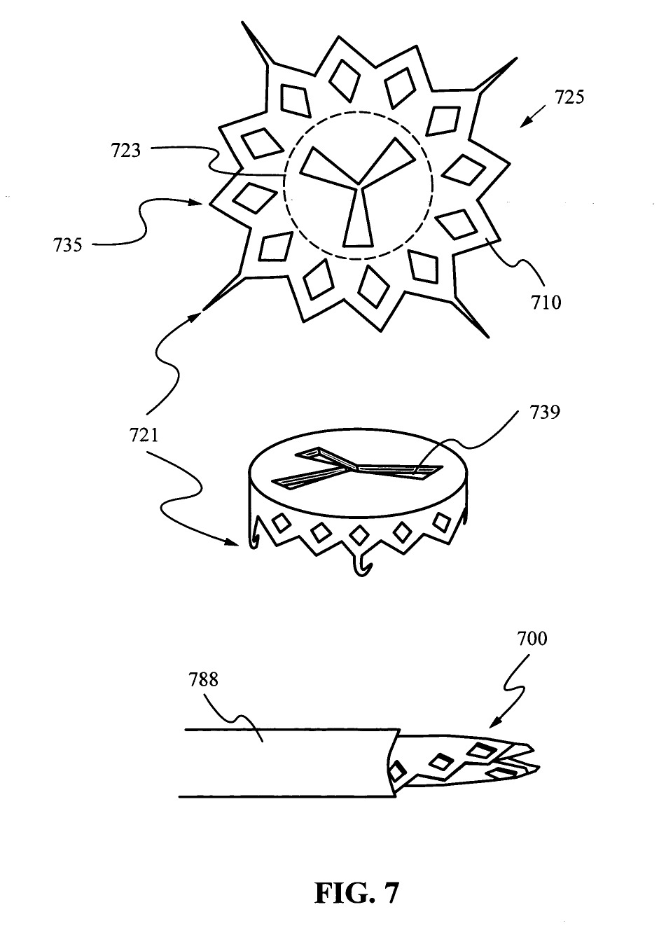

FIG. 7 shows a fifth embodiment of the implantable valve and steps of forming and delivering the same according to the present invention. Similar to the implantable valve 600 shown in FIG. 6, the frame structure 710 of the implantable valve 700 can be made from a substantially flat or tubular material. The frame structure 710 has an intricate pattern 725 with various openings 735, a plurality of anchoring sites 721, and a central opening 739. The openings 735 allows flexibility and could be used as attachment sites. The anchoring sites 721 could be used to anchor the implantable valve 700 without complex suturing. The dashed line 723 illustrates a desired diameter of the implantable valve 700.

The implantable valve 700 is worked, e.g., via bending and heat treatment, to its final shape. Optionally, surface treatment/modification may be applied to the implantable valve 700, after which it is coated with a biocompatible polymer utilizing one of the many methods described herein. Other coating methods known in the art may also be used.

The completed valve 700 may be delivered in a variety of methods. In some embodiments, it is rolled, folded, or otherwise reduced into a compact size and delivered through a catheter 788. Other folding techniques may also be used for catheter-based delivery and potentially anchoring the valve in place with minimal invasiveness.

In the embodiments described with reference to FIGS. 1-7, the substantially flat frame structure material is circular in general. The tapered end of each frame structure is initially positioned and formed at the center of the frame structure. Various cuts and openings, arranged periodically or non-periodically, spread outwardly from the center.

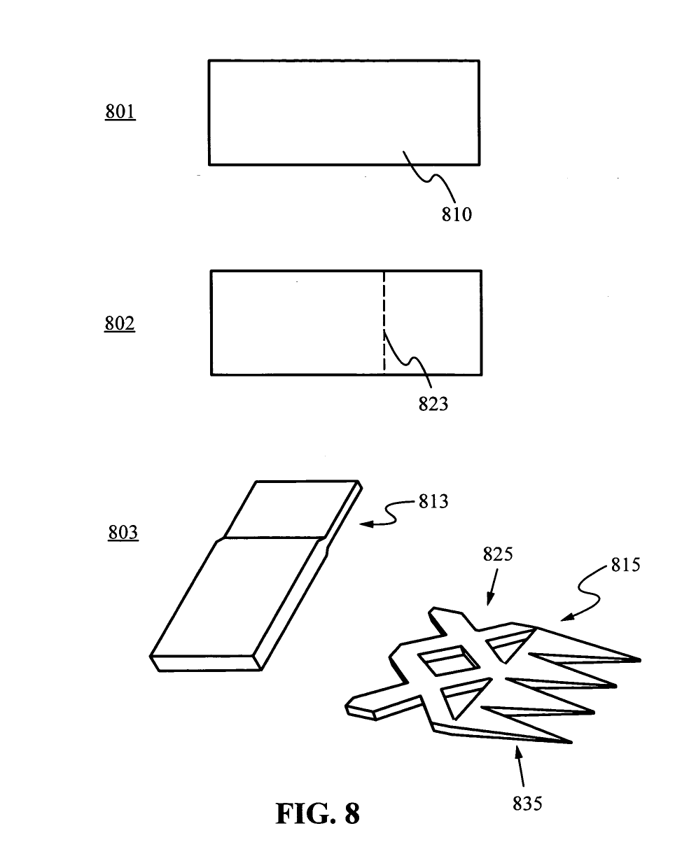

In the embodiments described below with reference to FIGS. 8-11, the substantially flat frame structure material is rectangular in general. The tapered end of each frame structure is formed at one edge thereof and the open end is correspondingly positioned at the opposite edge thereof. Various cuts and openings, arranged periodically or non-periodically, extending longitudinally from the open end edge to the tapered end edge, or vice versa. For example, in step 801, a rectangular frame structure material 810 is cut or molded to a desired size and/or configuration. Where applicable, in step 802, the frame structure material 810 is modified, e.g., by machining, molding, rolling, swaging, coining, scoring, cutting, etching, laser machined, etc., according to a reference line 823 to obtain a desired thickness profile. In step 803, the modified frame structure material 813 is further etched, stamped, or laser machined to produce a final frame structure 815 with a pattern 825 for forming an open end thereof and a plurality of tapered members 835 for forming a tapered end (tip) thereof. With an appropriate molding process and suitable material, the frame structure 815 could also be made in one step.

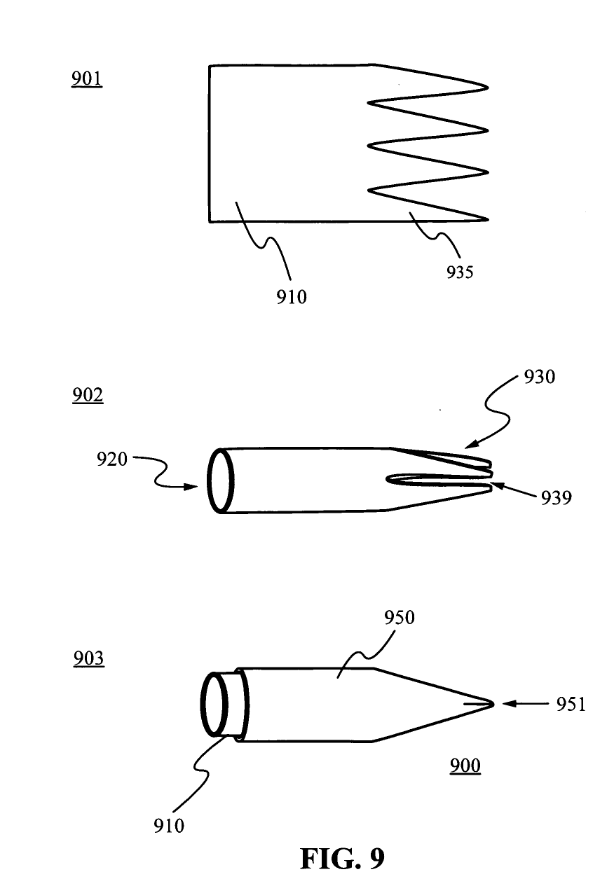

FIG. 9 shows a sixth embodiment of the implantable valve and steps of forming the same according to the present invention. Similar to the embodiment shown in FIG. 8, in step 901, the frame structure 910 is formed with a plurality of tapered members 935. The frame structure 910 may have openings (not shown) for allowing flexibility and/or anchoring segments (not shown) for reducing or eliminating complex suturing.

In step 902, the frame structure 910 is rolled into its final shape having an open end 920 and a tapered end 930. The frame structure 910 may be heat treated as described above.

In step 903, a biocompatible coating 950 is applied to the frame structure 910 via, for example, shrink-wrapping, dipping, adhesive bonding, laminating, effectively sealing gaps 939 between the tapered members 935 and enabling a threshold tip 951 to control or prevent fluid passage accordingly.

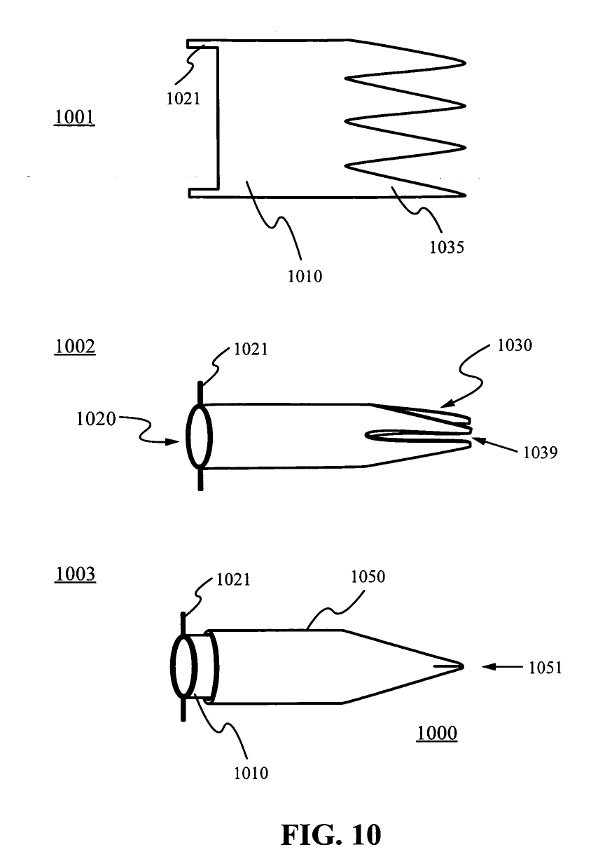

FIG. 10 shows a seventh embodiment of the implantable valve and steps of forming the same according to the present invention. Similar to the embodiment shown in FIG. 9, in step 1001, the frame structure 1010 is formed with a plurality of tapered members 1035. The frame structure 1010 has hooks 1021 for easy attachment to body tissue, thereby reducing or eliminating complex suturing. The frame structure 1010 may also have openings (not shown) to allow flexibility and/or expandability.

In step 1002, the frame structure 1010 is rolled into its final shape having an open end 1020 and a tapered end 1030. The frame structure 1010 may be heat treated as described above.

In step 1003, a biocompatible coating 1050 is applied to the frame structure 1010, effectively sealing gaps 1039 between the tapered members 1035 and enabling a threshold tip 1051 to control or prevent fluid passage accordingly.

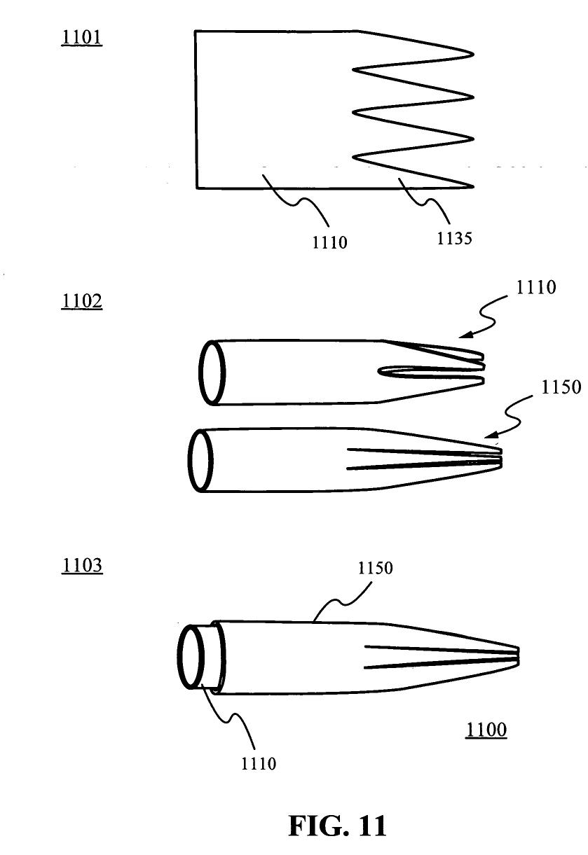

FIG. 11 shows an eighth embodiment of the implantable valve and steps of forming the same according to the present invention. Similar to the embodiments shown in FIGS. 9-10, in step 1101, the frame structure 1110 is formed with a plurality of tapered members 1135 for forming an open end and a tapered end thereof. The frame structure 1010 may have openings, attachment sites, and/or anchoring sites, which are not shown here for the sake of clarity.

In step 1102, the frame structure 1110 is rolled into its final shape. The frame structure 1110 may be heat treated as described above. A slightly larger tubular structure 1150 with long slits is similarly formed or made. The sleeve-like structure 1150 is characterized as soft, contrasting the more rigid frame structure 1110. The soft structure 1150 can be made from plastic or other flexible materials.

In step 1103, the rigid frame structure 1110 and the soft structure 1150 are assembled together by sliding the soft structure 1150 over the rigid frame structure 1110. Many known bonding materials and methods can be suitably employed and thus are not described herein. Although not shown, one skilled in art will appreciate that the soft structure 1150 can be made slightly smaller than the rigid frame structure 1110 so to fit snuggly inside thereof.

FIG. 12 shows a ninth embodiment of the implantable valve and steps of forming the same according to the present invention. In step 1201, the implantable valve 1200 is prepared from a hollow tube or rolled sheet having a desired diameter, thickness profile, material integrity, and so on. In step 1202, the tube or rolled sheet is cut, etched, or machined with a desired configuration, e.g., openings, attachment sites, anchoring sites, etc. In step 1203, the configured tube or rolled sheet is trimmed to a desired length, creating a frame structure 1210. The frame structure 1210 is turned to its final shape similar to the embodiments described above. The frame structure 1210 extends at least some amount radially inward.

In step 1204, a soft structure 1250 is made separately and assembled together with the frame structure 1210 to form the implantable valve 1200, similar to the embodiment shown in FIG. 11. Alternatively, the soft structure 1250 is molded directly onto the hard frame structure 1210 in step 1205.

Similar to other embodiments described herein, the hard frame structure 1210 could be made of non-metallic material, e.g., polycarbonate, polypropylene, or metallic material such as Nitinol superelastic or thermal actuation type. It could also be made of stainless steel, Elgiloy® or other shape memory materials.

Although the present invention and its advantages have been described in detail, it should be understood that the present invention is not limited to or defined by what is shown or described herein. As one of ordinary skill in the art will appreciate, various changes, substitutions, and alterations could be made or otherwise implemented without departing from the principles of the present invention.

For example, the customizable open end of the implantable valve can be tailored or otherwise configured in various ways to suit or adapt to different needs and application. The frame structure can be monolithically formed from a single piece of substantially flat material or in one step utilizing injection molding, insert molding, or other precision molding processes. Valves can be made from metallic pieces that have proper design and stiffness transitions to allow them to be extended farther radially inward. Valves can be made to allow dipping process to create the soft structure, i.e., the polymeric segment thereof. Valves can be made to utilize nanotechnology for surface modification. Valves can be made to utilize magnetic properties for positioning. Moreover, same designs could be obtained by a series of metallic or rigid plastic ribbons that are formed and bonded.

It is important to note that implantable valves according to the present invention can be readily scaled and are not limited by design. The embodiments implementing the present invention may vary in size and configuration depending on needs and applications and are limited only by the underlying manufacturing processes utilized.

Accordingly, the scope of the present invention should be determined by the following claims and their legal equivalents.

I claim:

1. An implantable valve, comprising:
a frame structure having a first portion characterized by an open end of said frame structure and a second portion characterized by a tapered end of said frame structure; and
a biocompatible coating or soft structure covering said frame structure or at least a portion thereof, wherein
said frame structure is monolithically made from a frame material; wherein
said open end has a desired diameter suitable for a particular implantation application;
wherein
said open end is configured with a plurality of openings to allow flexibility, expendability, and attachment to body tissue during said application; wherein
said tapered end comprises a plurality of tapered members that respectively gradually narrow to a tip and that are defined by a plurality of slits; and wherein
said biocompatible coating or soft structure seals or effectively covers said plurality of slits.

2. The implantable valve according to claim 1, wherein
said plurality of openings comprise a periodical or irregular pattern around said open end.

3. The implantable valve according to claim 1, wherein
said open end has periodical or irregular edges.

4. The implantable valve according to claim 1, wherein
said open end has anchoring sites for attaching said implantable valve to body tissue during said application.

5. The implantable valve according to claim 1, wherein
said frame material is substantially flat and has a thickness profile that enables said open end and said tapered end to have the same or different depths suitable for said application.

6. The implantable valve according to claim 5, wherein
said frame material is a slice from a tubular material.

7. The implantable valve according to claim 6, wherein
said tubular material has cavities, through holes, or a combination thereof that correspond to said plurality of openings.

8. The implantable valve according to claim 5, wherein
said frame material has a circular configuration in which said tapered end is initially formed at the center thereof with said plurality of slits spreading outwardly therefrom.

9. The implantable valve according to claim 5, wherein
said frame material has a rectangular configuration in which said tapered end is formed at one edge thereof and said open end is correspondingly form at the opposite edge thereof, with said plurality of openings and said plurality of slits, arranged periodically or non-periodically, forming longitudinally therebetween.

10. The implantable valve according to claim 1, wherein
said frame structure is shaped from said single material by way of stamping, molding, injection molding, coining, rolling, swaging, deep drawing, etching, laser machining, cutting, or a combination thereof.

11. The implantable valve according to claim 1, wherein
said frame structure is over a metallic or non-metallic mandrel that has higher heat-resistance than said frame structure and that does not react to said frame material.

12. The implantable valve according to claim 1, wherein
said frame structure is configured and said frame material is selected to allow said implantable valve to be rolled, folded, or reduced to a compact size small enough to be delivered percutaneously via a catheter.

13. The implantable valve according to claim 1, wherein
said frame material is a metallic material.

14. The implantable valve according to claim 13, wherein
said metallic material is superelastic or has heat recoverable shape memory.

15. The implantable valve according to claim 13, wherein
said metallic material is selected from the group consisting of stainless steel, Nitinol, Nitinol alloys, nickel-based alloys, cobalt-chromium-nickel alloys, Ni—Ti, Ni—Ti—Nb, Ni—Ti—Mo, Ni—Ti—V, Ni—Ti—Fe, Ni—Ti—Cu, Ni—Ti—Cr, shape memory alloys, and copper-based shape memory alloys.

16. The implantable valve according to claim 1, wherein
said single material is a synthetic resin made of a polymeric compound.

17. The implantable valve according to claim 16, wherein
said synthetic resin is selected from the group consisting of polycarbonate, polypropylene, and shape memory plastics.

18. The implantable valve according to claim 1, wherein
said biocompatible coating or soft structure is made from a material selected from the group consisting of silicones, polyvinyl, polyether-based polyamides, thermoplastic elastomers, polyurethane, polyethylene, anti-blood clotting coatings, anti-thrombogenic coatings, bioactive coatings, and heparin coatings.

19. The implantable valve according to claim 1, wherein
said biocompatible coating is applied to said frame structure by way of dipping, shrinking, bonding, laminating, insert molding, or nanotechnology molecular bonding.

20. The implantable valve of claim 19, wherein
surface of said frame structure is treated or modified utilizing nanotechnology.

21. The implantable valve according to claim 19, wherein
surface of said frame structure is treated or modified to enhance bonding between said frame structure and said biocompatible coating.

22. The implantable valve according to claim 21, wherein
said surface treatment or modification is selected from the group consisting of etching, plasma etching, polishing, sand blasting, plating, and a combination thereof.

23. The implantable valve according to claim 1, wherein
said first portion has a flexibility that is different from that of said second portion.

24. A method of making an implantable valve, comprising the steps of:
a) determining a two or three dimensional configuration of a three dimensional frame structure of said implantable valve, said configuration includes an open end and a tapered end;
b) scaling said configuration to a desired size suitable for a particular implantation application;
c) monolithically forming said frame structure or a plurality thereof from a frame material according to said configuration in step a) or step b); and
d) applying a biocompatible coating or soft structure to cover said frame structure or at least a portion thereof

25. The method according to claim 24, further comprising the steps of:
configuring and forming said open end with a plurality of openings to allow flexibility, expendability, and attachment to body tissue during said application; and
configuring and forming said tapered end with a plurality of tapered members that respectively gradually narrow to a tip and that are defined by a plurality of slits; wherein said biocompatible coating or soft structure seal or effectively covers said plurality of slits.

26. A medical apparatus made according to the method steps of claim 25.

27. A method of making an implantable valve, comprising the steps of:
a) determining a two or three dimensional configuration of a three dimensional frame structure of said implantable valve, said configuration includes an open end and a tapered end;
b) scaling said configuration to a desired size suitable for a particular implantation application;
c) applying a biocompatible coating or soft structure to a frame material; and
d) forming said frame structure or a plurality thereof from said frame material according to said configuration in step a) or step b).

28. The method according to claim 27, further comprising the steps of:
configuring and forming said open end with a plurality of openings to allow flexibility, expendability, and attachment to body tissue during said application; and
configuring and forming said tapered end with a plurality of tapered members that respectively gradually narrow to a tip and that are defined by a plurality of slits; wherein said biocompatible coating or soft structure seal or effectively covers said plurality of slits.

29. An implantable valve made according to the method steps of claim 28.
